(19) United States
(12) Patent Application Publication
(10) Pub. No.: US 2021/0307786 A1
(43) Pub. Date: Oct. 7, 2021

(54) Systems And Methods For Adjusting A Growing Rod

(71) Applicant: AMB Orthopedics, Inc., Mountain View, CA (US)

(72) Inventors: Lewis Tyson Ross, Franklin, OH (US); Casel Burnett, Walton, KY (US); Michael J. Wentz, Lake Forest, CA (US); Daniel Sands, Indianapolis, IN (US)

(21) Appl. No.: 17/274,359

(22) PCT Filed: Sep. 10, 2019
(86) PCT No.: PCT/US2019/050420

§ 371 Date: Mar. 8, 2021

Related U.S. Application Data
(60) Provisional application No. 62/824,365, filed on Mar. 27, 2019.
(60) Provisional application No. 62/729,096, filed on Sep. 10, 2018.

(51) Int. Cl. A61B 17/70; A61B 17/68; A61B 17/66; A61B 5/00

(52) U.S. Cl. CPC A61B 17/7016; A61B 17/688; A61B 17/663; A61B 2017/00221; A61B 5/4504; A61B 5/6878; A61B 5/0031

(57) ABSTRACT

A method for adjusting an adjustable implant includes broadcasting identification information from an adjustable implant implanted within a subject, receiving wirelessly with the adjustable implant, from a device external to the subject, an adjustment setting configured to be executed by the adjustable implant, receiving wirelessly with the adjustable implant, from a device external to the subject, an instruction to perform the adjustment setting, activating a motor on the adjustable implant to perform the adjustment setting, and sending, to a device external to the subject, a progress of the adjustment setting.

### BACKGROUND

Various aspects of the present invention relate generally to adjustable implants, which may include growing rods, and more specifically to wirelessly communicating with adjustable implants to perform an adjustment of the adjustable implant.

Growing rods are adjustable implants that are used for a variety of purposes, including as implants configured for gradually straightening a spine that is deformed (e.g., by scoliosis), or for growing in length along with the growth of an immature spine while also providing support for or influencing growth of that spine.

### SUMMARY OF THE INVENTION

In one embodiment of the present disclosure, a method for adjusting an adjustable implant includes broadcasting identification information from an adjustable implant implanted within a subject, receiving wirelessly with the adjustable implant, from a device external to the subject, an adjustment setting configured to be executed by the adjustable implant, receiving wirelessly with the adjustable implant, from a device external to the subject, an instruction to perform the adjustment setting, activating a motor on the adjustable implant to perform the adjustment setting, and sending, to a device external to the subject, a progress of the adjustment setting.

In another embodiment of the present disclosure, a non-transitory computer-readable medium for adjusting an adjustable implant, includes instructions stored thereon, that when executed on a processor, perform the steps of broadcasting identification information from an adjustable implant, collecting and storing or communicating an adjustment setting configured to be executed by the adjustable implant, collecting and storing or communicating an instruction to perform the adjustment setting, commanding a motor on the adjustable implant to perform the adjustment setting, and outputting a progress of the adjustment setting.

In yet another embodiment of the present disclosure, a system for adjusting tissue in a patient includes an adjustable implant including a first portion configured to couple to a first location in a subject, a second portion moveably coupled to the first portion and configured to couple to a second location in the subject, and a motor configured to move the first portion and the second portion relative to each other, and a device including a graphical user interface, wherein the adjustable implant is configured to broadcast identification information, and wherein the adjustable implant is configured to receive an adjustment setting from the device.

### BRIEF DESCRIPTION OF THE DRAWINGS

FIG. 1 is a plan view of system for adjusting tissue comprising an adjustable implant according to an embodiment of the present disclosure.

FIG. 2A is a flow chart illustrating a method for wirelessly adjusting a growing rod as seen from a external control device, according to an embodiment of the present disclosure;

FIG. 2B is a flow chart illustrating a method for wirelessly adjusting a growing rod as seen from the growing rod, according to an embodiment of the present disclosure;

FIG. 3A-F are screenshots of a graphical user interface to adjust a growing rod, according to an embodiment of the present disclosure; and

FIG. 4 is a block diagram illustrating a computing device that may be used to perform the method of FIG. 2A, according to an embodiment of the present disclosure.

FIG. 5 is a block diagram of a system for adjusting tissue in a patient according to an embodiment of the present disclosure.

FIGS. 6-9 schematically illustrate various embodiments of alternate sources of a driving element of an adjustable implant, according to embodiments of the present disclosure.

FIG. 10 illustrates an adjustable implant implanted in a femur, for lengthening thereof, according to an embodiment of the present disclosure.

FIG. 11 illustrates an adjustable implant implanted on a tibia, for lengthening thereof, according to an embodiment of the present disclosure.

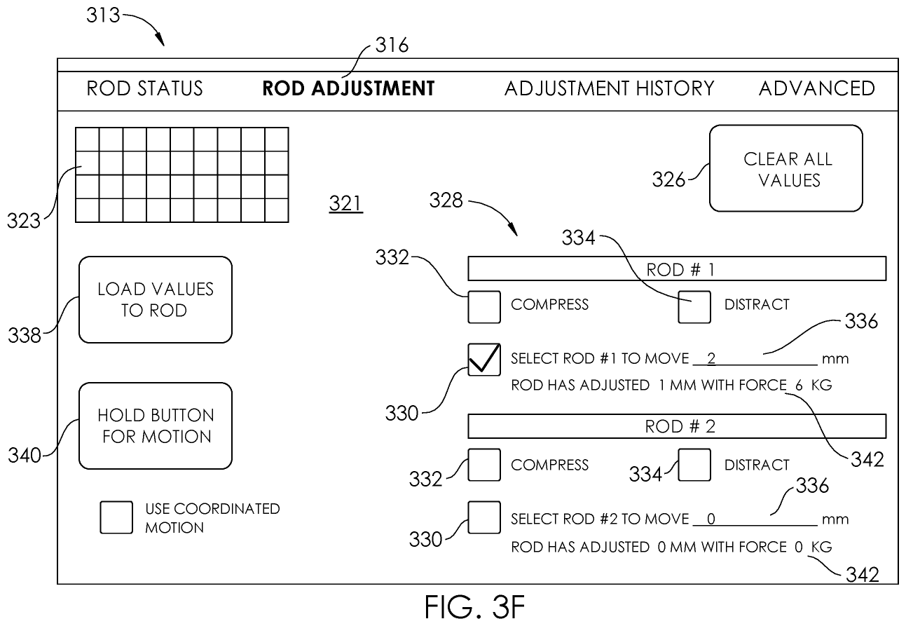

FIGS. 12 and 13 illustrate the straightening of a bone (femur), or how the curvature of a bone can be adjusted, using adjustable implants according to embodiments of the present disclosure.

FIGS. 14 and 15 illustrate curvature of a bone (femur) being adjusted using adjustable implants according to embodiments of the present disclosure.

FIGS. 16 and 17 illustrate the rotational orientation of portions of a bone (femur) being adjusted according to embodiments of the present disclosure.

FIG. 18 is a perspective view which shows the adjustable implants attached at osteotomy sites in the craniofacial skeleton.

FIG. 19 illustrates a cross-section of an embodiment of an adjustable implant comprising a suture anchor secured in the humerus of a rotator cuff surgery patient, according to an embodiment of the present disclosure.

FIG. 20 illustrates a humerus with a hole drilled for placement of an adjustable implant comprising a suture anchor in a rotator cuff patient.

FIG. 21 illustrates a tibia with a hole drilled for placement of an adjustable implant comprising a suture anchor in an anterior cruciate ligament patient.

### DETAILED DESCRIPTION OF THE ILLUSTRATED EMBODIMENTS

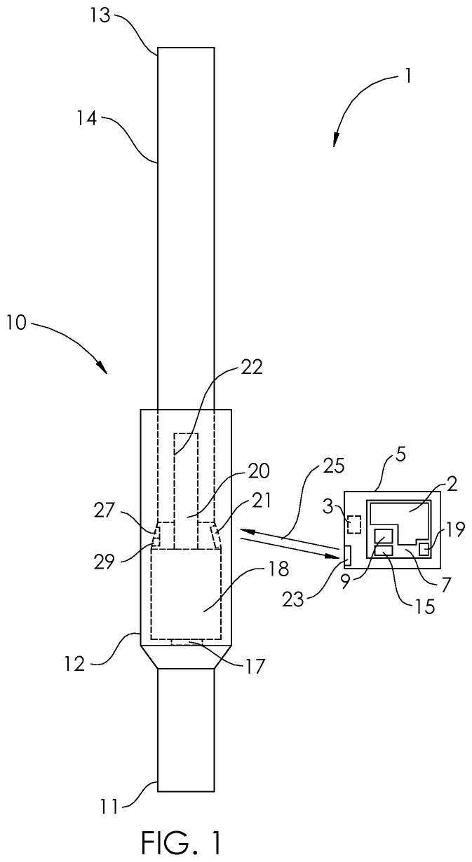

Systems and methods are disclosed herein for adjusting an adjustable implant, such as a growing rod, that is coupled to a patient (internally or externally). A system for adjusting tissue in a patient 1 comprising an external control device 5 and an adjustable implant 10 is illustrated in FIG. 1. The external control device 5 is configured to non-invasively adjust the adjustable implant 10. The adjustable implant 10 comprises a first implant portion 12 and a second implant portion 14, the second implant portion 14 non-invasively displaceable in relation to the first implant portion 12. The first implant portion 12 is secured to a first portion of tissue (e.g., bone or soft tissue) and the second implant portion 14 is secured to a second portion of tissue (e.g., bone or soft tissue) within a patient. A motor 18 is operable to drive a lead screw 20 within a female threaded portion 22 to cause the first implant portion 12 and the second implant portion 14 to displace relative to one another and thus change the length of the implant, and thus move the first portion of tissue and the second portion of tissue in relation to each other. Alternatively, the motor 18 may rotate a nut (not shown) in relation to a rotationally static threaded rod to cause the first implant portion 12 and the second implant portion 14 to displace relative to one another and thus change the length of the implant, and thus move the first portion of tissue and the second portion of tissue in relation to each other. In some embodiments, the adjustable implant may comprise one or more embodiment described in U.S. Pat. No. 8,585,740 entitled “Automated Growing rod Device,” issued Nov. 19, 2013, which is hereby incorporated by reference in its entirety for all purposes. The motor 8 may comprise an electric motor, such as a brushed or brushless motor, a DC motor, a stepper motor, a servo motor, a linear motor, or an ultrasonic or piezo motor, including an inertia motor, a resonance motor, or a piezo-walk drive.

There are a large number of configurations for adjustable implants, including growing rods. A “growing rod” device is a type of adjustable implant that is configured to be mounted to a long bone or to the spine of a patient, a that is configured to have its overall length extended (or distracted) or reduced (or compressed) in situ. Growing rod devices have been developed for implantation in the spine of growing patients to correct an abnormal curvature of the spine, such as scoliosis. In certain devices of this type, a rod assembly is progressively lengthened to match or even surpass a child's vertical growth in the spine, and to maintain or increase correction of the abnormal curvature. In some embodiments, a pair of growing rods may be implanted, one on each side of the spine at least along the area of deformation or abnormal curvature. The rod or rods may be lengthened yearly, every six months, every three months, every two months, monthly, or even weekly or daily. Adjustable implants may be utilized to treat a number of different maladies, including, but not limited to early onset scoliosis, adolescent idiopathic scoliosis, limb-length discrepancy, cranio-maxillofacial deformity, soft tissue tightness, or soft tissue laxity, including the adjustment of ligaments, such as the ACL, or muscles, such as eye muscles in a strabismus patient, or tendons and muscles, such as the rotator cuff.

A “external control device” is a device configured to be used externally to a patient to at least partially control the operation of an adjustable implant that is implanted within the patient.

In some embodiments, a user (e.g., a physician, medical professional, family member, friend, or even the patient) interacts with a graphical user interface (GUI) 7 on an external control device 5 (FIG. 1) to send instructions to the adjustable implant (e.g., growing rod) to perform an adjustment. It should be understood that although a growing rod may be referenced in describing a particular system, the external control devices described herein may be utilized in a similar manner with a number of different adjustable implants that are not necessarily growing rods. For example, adjustable implants that do not “grow” in length, and/or adjustable implants that are not “rods” may be considered as such. An example of an adjustable implant that is not a growing rod is a restricting ring or band that may be adjusted to decrease or increase its diameter, and may be placed over a tract within a patient to increase or adjust restriction on the tract. For example, the device may be configured to adjust restriction on an esophagus, stomach, anus, or urethra. In some embodiments, the adjustable implant may comprise one or more embodiment described in U.S. Pat. No. 7,238,191 entitled “Surgical ring featuring a reversible diameter remote control system,” issued Jul. 3, 2007, which is hereby incorporated by reference in its entirety for all purposes. The adjustable implant/growing rod 10 includes a transceiver 21 and the external control device 5 includes a transceiver 23 for two-way communication (signals 25) between the growing rod 10 and the external control device 5. The transceiver 21 may be carried on, inside of, or adjacent the growing rod 10. The transceiver 23 may be carried on, inside of, or adjacent the external control device 5. When the user sends a start instruction from the GUI 7 of the external control device 5, the growing rod 10 starts a motor 18 to perform the adjustment, for example, the motor 18 carried by the adjustable implant 10 that is configured to move a first end 11 and a second end 13 in relation to each other. When the first end 11 and the second end 13 are moved apart from each other, it is considered lengthening, and then the first end 11 and second end 13 are moved toward each other, it is considered shortening. The growing rod 10 may comprise two elongate, telescopically-coupled sections, with the motor 18 configured to increase or decrease the overall length of the growing rod 10 by longitudinal movement between the two sections, as described relative to the adjustable implant 10 of FIG. 1. Commonly, growing rods, when used in pediatric patients for scoliosis treatment, are lengthened, either periodically or continuously, to correspond with normal growth in an immature patient. Once the motor 18 is initiated to perform the adjustment, if the user sends a stop or pause instruction, the growing rod 10 will stop the motor 18 until told to resume. When the growing rod 10 finishes the adjustment, the growing rod 10 communicates to the external control device 5 that the adjustment is complete and then stores a record in a database on the growing rod itself.

The systems comprising growing rods 10 of the present disclosure and the methods for adjusting the growing rods 10 exhibit several advantages over existing solutions. For example, the growing rods 10 disclosed herein are adjustable without requiring the patient to go through a surgery for the adjustment. Further, by sending a precise length to be adjusted, there is not a fear of over adjusting or under adjusting the growing rod 10 during an adjustment, which provides a benefit over certain magnetically controlled growing rods that do not have direct feedback of adjustment length.

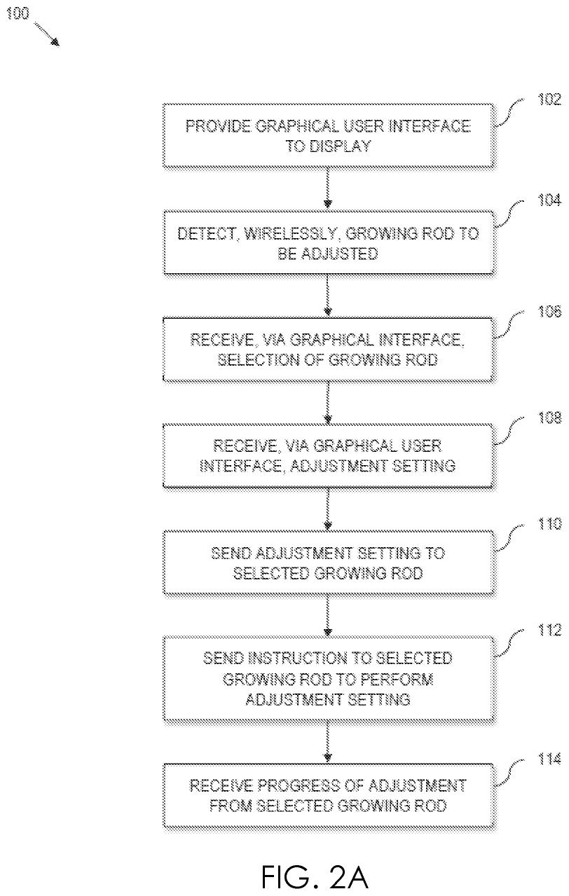

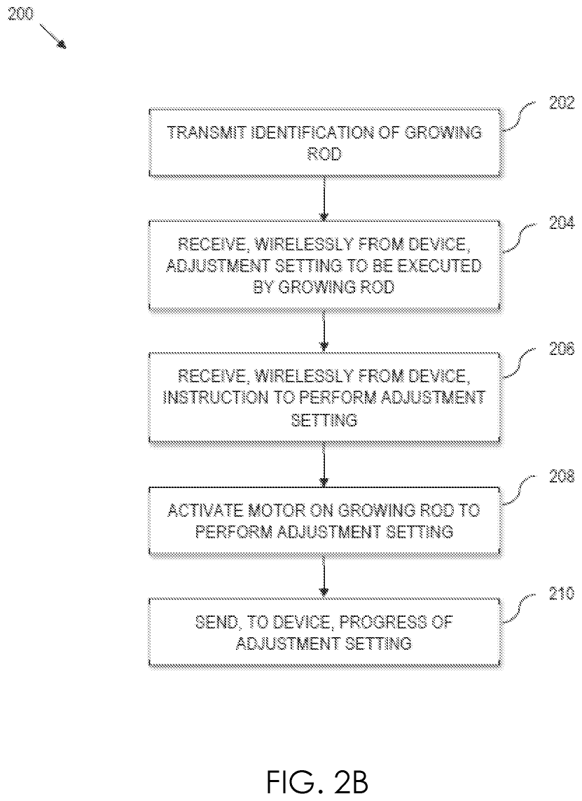

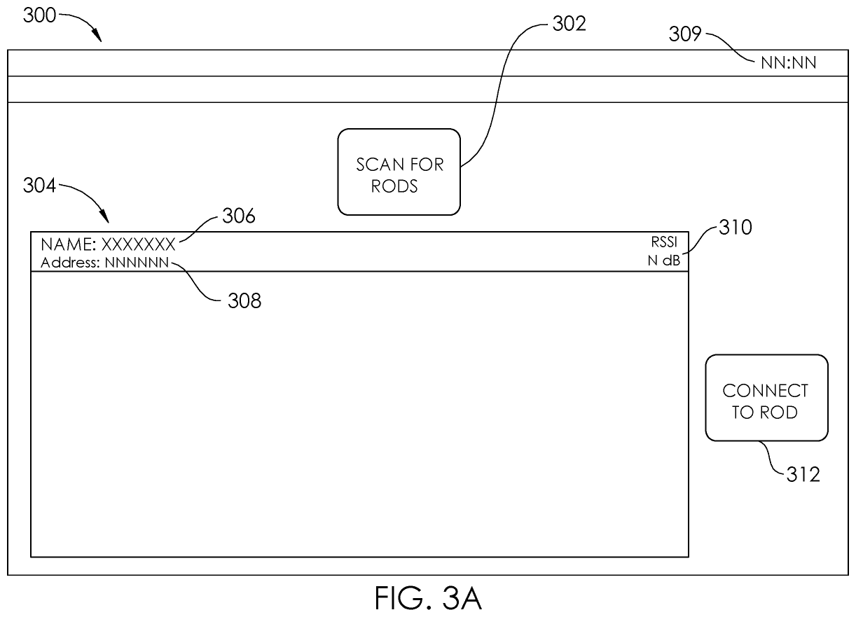

As shown in FIG. 2A, a method 100 for adjusting a growing rod 10 is shown. At step 102, a graphical user interface (GUI) 7 is provided as a display of an external control device 5 that is configured for allowing user control of the method 100. The GUI 7 is discussed in greater detail in reference to FIGS. 3A-F below. The external control device 5 may be any device with a display 2 and a processor 3. In some embodiments, the processor 3 may comprise a microprocessor. The external control device 5 may comprise a laptop computer, a desktop computer, a tablet computer, a smart device, such as a smartphone, etc., or combinations thereof.

At step 104, the external control device 5 wirelessly detects a growing rod (e.g., adjustable implant 10) that needs to be adjusted. In some cases, the growing rod 10 may need to be lengthened to match growth that has occurred or is occurring or about to occur in the patient. In some cases, the growing rod 10 may need to be lengthened to increase a distraction force, which may be described as the force attempting to push two different portions of an anatomy apart from each other. In some cases, the growing rod 10 may need to be shortened to decrease a distraction force, or even to eliminate a distraction force. In some cases, the growing rod 10 may need to be shortened to apply a compressive force, which may be described as the force attempting to push two different portions of an anatomy toward each other. The external control device 5 may wirelessly detect the growing rod 10 in the following manner. The growing rod 10 transmits identification information (e.g., identification of the particular growing rod 10, such as a serial number or another identifier) via any wireless technology (e.g., wireless fidelity (WiFi), Bluetooth®, ultra-wide band (UWB) protocol, etc.), and the external control device 5 reads the identification information and displays some or all of the identification information on or by use of the GUI 7. In some embodiments, the display may be entirely visual, but in other embodiments, the identification information may be communicated by audio means, for example, a computer voice or recorded voice. In some embodiments, multiple growing rods 10 are detected, and their associated identifications are aggregated into a list of identifications for the growing rods 10. Then, this list is displayed via the GUI 7.

Further, the external control device 5 may receive other information from the growing rod 10. For example, the growing rod 10 may send the patient's name, weight, height, sitting height, Cobb angle, date of birth, Risser sign, Tanner stage, age of at menarche, etc. As another example, the growing rod 10 may send a history log of all (or a subset of) adjustments performed by the growing rod or growing rods 10.

Moreover, the external control device 5 does not need to communicate with the growing rod 10 directly. For example, the growing rod 10 may be Internet enabled and may communicate over the Internet or some other network. The external control device 5 is configured to access a device on the Internet (or network) that can communicate as an intermediary with the growing rod 10 As another example, the external control device 5 may communicate with another external control device 5 as an intermediary to communicate with the growing rod 10. Thus, the user may be remote from the growing rod 10 (e.g., in a different room, a different building, a different city, county or state, a different country, etc.).

At step 106, the external control device 5 receives a selection of a particular growing rod 10 displayed on the external control device 5. For example, as discussed above, the identification information/identifier of the growing rod 10 is displayed to the user via the GUI 7. The user can then select the growing rod 10. In embodiments that aggregate several growing rods 10 into a list, the user selects the identification information of one of the growing rods 10 in the list. Depending on the wireless protocol, the external control device 5 sets up communication with the selected growing rod 10. For example, if the wireless protocol is Bluetooth, then the external control device 5 pairs with the selected growing rod 10. It is common for patients to have one or two implanted growing rods 10, or sometimes more. When there is more than one implanted growing rod, the ability to select the appropriate growing rod 10 is particularly useful. In some cases, the user will see both of the implanted growing rods' 10 identification information on the GUI 7, and will first select the particular growing rod 10 that is desired for adjustment, or that is desired to be adjusted first.

At step 108, the external control device 5 receives an adjustment setting from the user via the GUI 7. In some embodiments, the adjustment setting includes a direction setting and a length setting. For example, the adjustment setting may have a direction of distraction and a length. In one representative case, the direction can be an increase in length and the length of increase (e.g., distraction) can be three millimeters. In another representative case, the direction can be a decrease in length, and the length decrease can be two millimeters. In some embodiments, the adjustment setting may include multiple adjustments, including, a first adjustment and a second adjustment, or a first, second and third adjustment. In some embodiments, the multiple adjustments may be scheduled periodically, for example, one adjustment each month, one adjustment each week, one adjustment each day, or even multiple adjustments each day. In some embodiments, the adjustment may comprise a single, continuous adjustment, for example a slowly increasing length, at a velocity of three millimeters per month. In some cases, the adjustment (e.g., length per time) may be calculated from at least some of the patient data, e.g., weight, height, sitting height, Cobb angle, date of birth, Risser sign, Tanner stage, age of at menarche. If the one or more indicators from the patient data represent a patient that is likely to be growing at significantly faster rate, the adjustment may include a cooperatively faster distraction length increase rate. If the one or more indicators from the patient data represent a patient that is likely to be growing at significantly slower rate, the adjustment may include a cooperatively slower distraction length increase rate.

At step 110, the external control device 5 wirelessly sends the adjustment setting to the selected growing rod 10. For example, the external control device 5 can send the direction and the length of the adjustment to the growing rod 10. The external control device 5 can also send the adjustment velocity (e.g., mm per minute, mm per hour, etc.) to the growing rod 10.

At step 112, the external control device 5 sends an instruction to the growing rod 10 to perform an adjustment according to the according to the one or more sent adjustment settings. For example, the GUI 7 may include a button 9 (FIG. 1) for the user to push to start the adjustment. Thus, the external control device 5 receives the command to start the adjustment from the user via the GUI 7 and sends an instruction to the growing rod 10 to start the adjustment per the adjustment setting. The growing rod 10 then activates a motor 18 in the growing rod 10 to start the adjustment according to the adjustment settings. In some embodiments, the external control device 5 receives an instruction from the user via the GUI 7 to stop or pause the adjustment before the adjustment is complete. In such a case, the external control device 5 sends an instruction to the growing rod 10 to stop the motor 18. The instruction from the user to pause or stop the adjustment may be received through the same button 9 on the GUI 7, or by a different button 15. For example, a single button may be present, and when the user presses the button, the adjustment starts. However, when the user lets go of the button, an instruction to stop or pause the adjustment is sent. When the button is pressed again, the adjustment resumes. Alternatively, there may be separate buttons to start/proceed and stop/pause the adjustment.

Moreover, instead of one instruction to start and another to stop, the external control device 5 may send a series of instructions at a set interval that instructs the growing rod 10 to perform a subset of the complete adjustment. If one of the instructions is not received, then the growing rod 10 does not perform that subset and waits for the instruction for that subset. For example, if the user presses the button, the external control device 5 keeps sending instructions to proceed to the growing rod 10, but when the user lets go of the button, then the external control device 5 does not send an instruction to the growing rod 10.

At step 114, the external control device 5 receives a progress of the adjustment from the selected growing rod 10. For example, once the adjustment completes (e.g., once the growing rod 10 extends three millimeters), then a message indicating the completion of the adjustment is sent from the growing rod 10 to the external control device 5. As another example, while the adjustment is progressing, sensor data from a sensor 17 (FIG. 1) on or associated with the growing rod 10 (e.g., gyroscope, force sensor, encoder, etc.) may be sent from the growing rod 10 to the external control device 5, which can then display the sensor data on the GUI 7 for the user to access. Alternatively, the adjustment setting may include a force target instead of a length target. For example, the adjustment setting when initiated may cause the motor 18 to increase the length of the growing rod 10 until the sensor 17 determines that a target force has been achieved. In some cases, a target force of 30 pounds (force) may be the target. In other cases, a target force may exist, but a maximum length increase may also be included in the adjustment setting. Thus, if the desired force is 25 pounds (force) and the maximum length increase is five millimeters, then the motor 18 will be instructed per the adjustment setting to increase the length of the rod until either 25 pounds is sensed by the sensor 17, or an increase of five millimeters is reached, whichever comes first. In other embodiments, additional quantities may be chosen, instead of length or force, or along with length and/or force. For example, the additional quantities may include motor temperature, motor duty cycle time, available battery capacity/charge level, clock time, Cobb angle (if being measured, e.g., by an implanted goniometer) or other parameters. The GUI 7 may then display the progress of the adjustment for each growing rod 10 on the display 2. The GUI 7 may display the current length of the growing rod 10, and/or the total change in the growing rod 10 length change during the adjustment, and/or the last measured distraction force, and/or other parameters of interest.

FIG. 2B is a flow chart illustrating a method 200 for adjusting a growing rod 10 in reference to and from the perspective of the growing rod 10, itself. At step 202, the growing rod 10 wirelessly broadcasts an identifier (identification information) for an external control device 5 to detect the growing rod 10. The growing rod 10 may broadcast the identifier using any wireless technology, as those described above. Furthermore, the broadcast may be at a set interval (e.g., one hundred milliseconds) or through use of some other formula. For example, the external control device 5 may have a button 19 to wake up a sleeping growing rod 10, and the growing rod 10 will broadcast the identifier in response to the user pressing the button 19. The external control device 5 then sets up a communication with the growing rod 10 depending on the wireless protocol used (e.g., the device pairs with the growing rod 10 if Bluetooth is used).

Furthermore, the growing rod 10 may broadcast or otherwise transmit information stored on the growing rod 10. For example, the growing rod 10 may transmit battery information, sensor status, memory usage, patient identifiers, patient weight, patient height, patient age, adjustment history, etc.

At step 204, the growing rod receives an adjustment setting to be executed from the external control device 5. As discussed above, the adjustment settings may include a direction setting and a length setting, and even an adjustment velocity, or an average adjustment velocity. The growing rod 10 uses these adjustment settings to determine which direction to turn a rotor of a motor 18 and for how long of a period of time to do so. The adjustment settings may also be stored in a database on the growing rod 10 itself.

At step 206, the growing rod 10 receives an instruction to perform an adjustment according to the adjustment setting(s), and at step 208 activates the motor 18 to perform the adjustment according to the adjustment setting. For example, the growing rod 10 will continue to perform the adjustment until the growing rod 10 completes the adjustment or receives an instruction to stop. As another example, the growing rod 10 will activate the motor 18 up on receiving the instruction but only for a subset of the complete adjustment. When the growing rod 10 receives another instruction to proceed, then the growing rod 10 will complete another subset of the complete adjustment. This continues as long as the growing rod 10 receives instructions to proceed or the adjustment is completed. In some cases, the instruction to perform the adjustment may comprise an instruction to start the adjustment setting. This instruction causes the motor 18 to activate. A controller 27 (e.g., microcontroller) carried on the growing rod 10 may directly activate the motor 18 to achieve this. The controller 27 may comprise a processor that is configured to execute a non-transitory computer-readable medium comprising instructions. The instructions may include the instruction to activate the motor 18, to temporarily pause the motor 18, to stop the motor 18, or to reactivate the motor 18. In some cases, the instruction to perform the adjustment may comprise an instruction to pause the adjustment setting. This instruction causes the motor 18 to temporarily stop its operation. In some cases, the instruction to perform the adjustment may comprise an instruction to resume the adjustment setting. This instruction causes the motor 18 to reactivate. In some cases, the instruction to perform the adjustment may be received from a different device than the device from which the adjustment setting was received. For example, the adjustment setting may be received from a first tablet computer (e.g., iPAD®, etc.) and the instruction to perform the adjustment setting may be received on a second tablet computer. Alternatively, the adjustment setting may be received from a tablet computer and the instruction to perform the adjustment per the adjustment setting may be received by a smartphone. In other cases, both the adjustment setting and the instruction to perform the adjustment setting are both received from the same device. They may be received at different times or received at the same time.

At step 210, the growing rod 10 sends a progress of the adjustment to the external control device 5. For example, once the adjustment completes (e.g., once the growing rod 10 extends three millimeters), then a message indicating the completion of the adjustment is sent from the growing rod 10 to the external control device 5. As another example, while the adjustment is progressing, sensor data (e.g., gyroscope, force sensor, encoder, etc.) may be sent from the growing rod 10 to the external control device 5, which can then display the sensor data on the GUI 7 for the user to access. In some cases, the progress may be sent to the same device from which the adjustment setting was received, and/or to the same device from which the instruction to perform the adjustment setting was received. In some cases, the progress may be sent to a different device than that or those from which the adjustment setting and the instruction to perform the adjustment setting were received. The progress of the adjustment performed on the growing rod 10 may then be stored in memory 29 on the growing rod 10.

FIGS. 3A-F illustrate screenshots of the GUI 7 associated with the methods 100, 200 above. FIG. 3A is a screenshot of a first screen 300 that is configured to allow a user to wirelessly couple to a growing rod 10. Any of the buttons in each screen/screenshot may be configured as touch sensitive (e.g., capacitive, resistive, optical, surface acoustic wave, electromagnetic). In other embodiments, any of the buttons may be replaced by voice recognition-capable controls, whether they are visually presented on the screen 300 or not. A scan for rods button 302 is available for the user to press and is configured to enable the external control device 5 to detect growing rods 10 that are transmitting identification (e.g., identifiers). Any growing rods 10 that are found are placed in a list 304 along with an identifier 306, a network address 308 (or other identifier for other wireless protocols), and a signal integrity indicator 310, such as a received signal strength indicator (RSSI). The identifier 306 may include a particular model number and/or a serial number. The user can select one or more of the rods listed and press a connect to rod button 312 to connect to the selected growing rods. In an example, the user selects the only growing rod 10 in the list and connects to that rod by pressing the connect to rod button 312. Though only one growing rod 10 is listed in the list 304 of FIG. 3A, there may be several growing rods listed. In some cases, the list 304 may contain all of the growing rods 10 from a single patient. In some cases, the list 304 may actually contain the growing rods 10 from a single patient, and the growing rods 10 from another patient, for example, a second patient within the same clinic that is within the vicinity of transmission. The first screen 300 may also include a time stamp and date stamp 309.

FIG. 3B illustrates a second screen 313 configured to be displayed when the external control device 5 couples to a growing rod 10, the screen 313 including tabs for rod status 314, rod adjustment 316, adjustment history 318, and advanced options 320. As shown in FIG. 3B, the rod status tab 314 is active and shows a first display (sub-screen) 319 including indications for different aspects 322 of the coupled growing rod 10 and associated patient information 324. The aspects 322 may include CPU battery status (good/bad/low), motion battery (e.g., motor battery) status (good/bad/low), sensor status (active/inactive/correct range/clipped range), available memory, used memory, or other operational parameters. The patient information 324 may include patient record number, patient name, patient weight, patient age or birthdate, patient height, patient sitting height, Cobb angle, Risser sign, Tanner stage, age of at menarche, or other parameters.

When the user selects the rod adjustment tab 316, the display (sub-screen) 321 illustrated in FIG. 3C appears, which includes a button 326 to clear all previous adjustment values. Further, there is a space 328 to enter adjustment settings: selecting a rod 330, compression 332 (or shortening) or distraction 334 (or lengthening) (i.e., a distraction direction), and a particular length to adjust 336. A length change in a positive distraction direction (+Z) may be described as distraction or lengthening and a length change in a negative distraction direction (−Z) may be described as compression or shortening. Number or letter keys 323 may be used to enter text, or voice-activated controls may automatically enter the data from user instructions. When the user enters the adjustment settings and presses a load values button 338, the adjustment values are sent to the selected growing rod(s) 10. As shown in FIG. 3C, a compression of one millimeter will be sent to the selected growing rod 10 when the load values button 338 is pressed. An additional message may be displayed on the display 2 of the GUI 7, such as “ADJUSTMENT COMPLETED.”

After the adjustment settings are loaded, an activation button 340 is available for the user to press. When the user presses the activation button 340, the growing rod 10 activates its motor 18 to perform an adjustment according to the adjustment settings. In some embodiments, the activation button 340 may include a visual (and/or audio) command to the user, such as “HOLD BUTTON FOR MOTION.” As described above, the adjustment may be paused before it is completed. The activation button 340 may be configured so that it toggles between an active and an inactive condition. Alternatively, the activation button may be configured to be active only if contacted. For example, if the user lets go of the activation button 340, then the adjustment stops until the user presses the activation button 340 again.

A progress of the adjustment 342 is shown. The progress 324 may be updated periodically during the adjustment. Furthermore, when the adjustment is completed, the progress is updated as shown in FIG. 3D. The progress may include the length successfully adjusted as well as the force (e.g., compressive force) on the rod, currently measured by a sensor 17 (e.g., force sensor, strain gauge, piezoelectric element, etc.).

FIG. 3E shows the display (sub-screen) 325 when the adjustment history tab 318 is selected. The data for the adjustment history 331 is stored on the growing rod 10 and sent to the external control device 5 for display, for example, on the display 2 of the GUI 7. The data for the adjustment history 331 may be sent at any time (e.g., upon connection, before connection, when the adjustment history tab 318 is active, etc.). The adjustment history 331 may include the date or time of adjustments (distractions, compressions) or assessments (snapshots), the type of adjustment (distraction, compression), the length of each adjustment and the force obtained by the sensor 17, to total lifetime adjustment of the growing rod 10, the rod position, a physiological angle (e.g., Cobb angle or other angle), patient height, patient sitting height, patient weight, or other demographics. Patient data 327 and growing rod information 329 are listed on the display (sub-screen) 325. As shown in FIG. 3E, the patient data 327 represents the patient data at the time of the implantation surgery, but in alternative embodiments, the patient data 327 may represent the updated or current patient data.

FIG. 3F illustrates the rod adjustment tab 316 when the external control device 5 is coupled to more than one growing rod 10. The user may provide adjustment settings for more than one growing rod 10 (Rod #1, Rod #2). Furthermore, the user can select all or a subset of the growing rods 10 to send adjustment settings or to adjust simultaneously, or serially. In some cases, the multiple growing rods 10 (e.g., two rods) may be commanded to adjust at the same time. For example, rod #1 and rod#2 may each increase two millimeters, or rod #1 may increase two millimeters while rod #2 increases 3.5 millimeters, or rod #1 may increase three millimeters while rod #2 decreases one millimeter, etc. In other cases, rod #1 may increase two millimeters, after which rod #2 increases two millimeters. In other cases, rod #1 may increase two millimeters, after which rod #2 increases two millimeters, after which rod #1 distracts until a set force reading is observed on the sensor 17 of rod #1, after which rod #2 distracts until a set force is observed on the sensor 17 of rod #2. When the select rod 330 box of rod #1 is selected (as shown in FIG. 3F), the activation button 340 becomes configured to actively operate the adjustment of rod #1. When the select rod 330 box of rod #2 is selected, the activation button 340 becomes configured to actively operate the adjustment of rod #2.

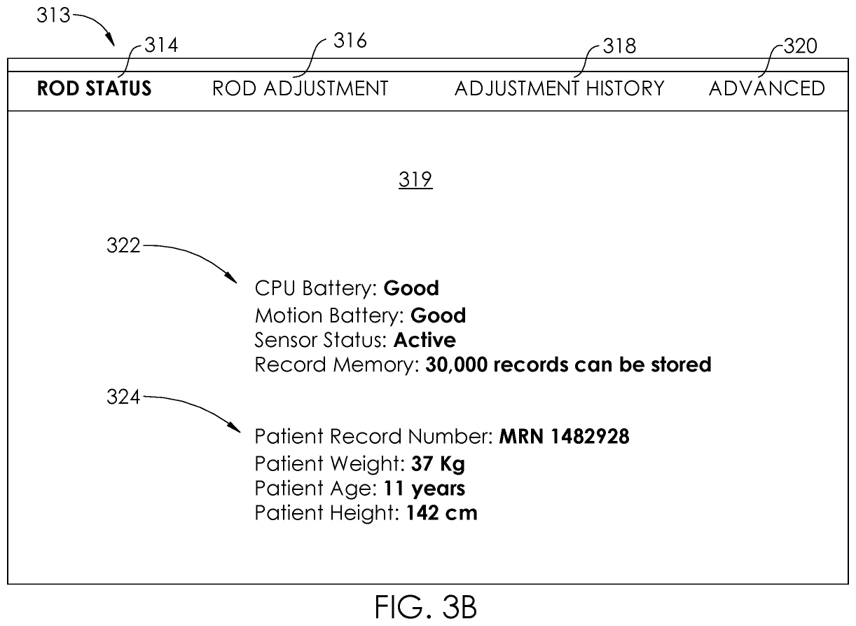

Referring to FIG. 4, a hardware data processing system 400 is depicted in accordance with the present disclosure. The data processing system 400 may comprise a symmetric multiprocessor (SMP) system or other configuration including a plurality of processors 410 connected to system bus 430. Alternatively, a single processor 410 may be employed. Any of the processors 410 may comprise a microprocessor. Also connected to the system bus 430 is local memory 420, e.g., RAM and/or ROM. An I/O bus bridge 440 interfaces the system bus 430 to an I/O bus 450. The I/O bus 450 is utilized to support one or more buses and corresponding devices, such as storage 460, removable media storage 470, input and output devices 480, network adapters 490, other devices, or combinations thereof, etc. For instance, a network adapter 490 can be used to enable the data processing system 400 to communicate with other data processing systems or remote printers or storage devices through intervening private or public networks.

The memory 420, storage 460, removable media storage 470 or combinations thereof can be used to store program code that is executed by the processor(s) 410 to implement any aspect of the present disclosure described and illustrated in the preceding figures.

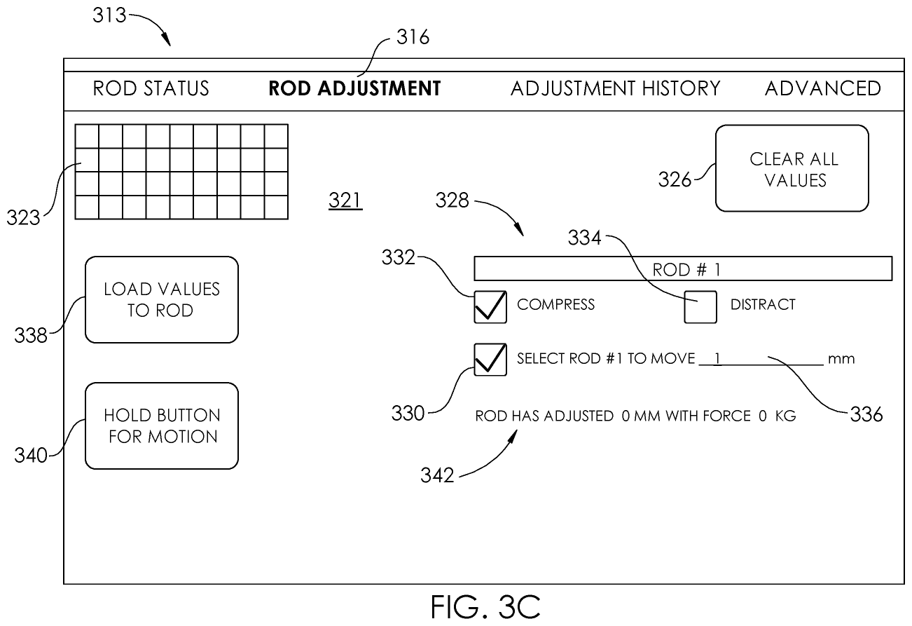

A system for adjusting tissue in a patient 500 is illustrated in FIG. 5. The system 500 comprises two adjustable implants 502, 504, shown implanted within a subject 506 having a scoliotic spine 508. The adjustable implants 502, 504 are non-invasively adjustable to treat the scoliosis of the subject 506. An external control device 510 having a graphical user interface (GUI) 512 is configured to non-invasively control the adjustment of the adjustable implants 502, 504. The adjustable implants 502, 504 each comprise a first implant portion 514, 516 and a second implant portion 518, 520, the second implant portions 518, 520 non-invasively displaceable in relation to the first implant portions 514, 516. The adjustable implants 514, 516 comprise dual growing rods configured to be distracted along with the natural growth of the immature subject 506, to maintain a distraction force on the spine 508, continuing treatment of the scoliosis. The first implant portions 514, 516 are secured to a first vertebral body 522 with pedicle screws, hooks, or other types of instrumentation. The second implant portions 518, 520 are secured to a second vertebral body 524 with pedicle screws, hooks, or other types of instrumentation. Each of the two adjustable implants 502, 504 comprise a motor 526, 528 (similar to motor 18 of FIG. 1) that is operable to cause the first implant portions 514, 516 to be displaceable in relation to the second implant portions 518, 520, to change the length of the implant, and thus move the first vertebral body 522 and the second vertebral body 524 in relation to each other.

The external control device 510 may comprise a laptop computer, a desktop computer, a tablet computer, a smart device, such as a smartphone, etc., or combinations thereof. The external control device 510 is configured for two-way communication (signals 530) with the adjustable implants 502, 504, in a similar manner described in relation to the external control device 5 and adjustable implant 10 of FIG. 1. The GUI 512 is configured to run the screens 300, 313 described in relation to FIGS. 3A-3F. A wearable device 532 comprising a wrist band is configured to be worn on a limb of a patient, or even on the limb of a parent, family member, friend, or health care professional. As shown in FIG. 5, the wearable device 532 is secured to the right wrist of the subject 506 and is configured to be used as an intermediary device. The wearable device 532 incorporates a controller 534 and a user interface 536. The user interface 536 may comprise a GUI, but may in some embodiments comprise only one or more buttons configured to be operated by the user. The user may be the subject 506 wearing the wearable device 532, or may be a person other than the subject 506. Furthermore, the wearable device 532 may be configured to be worn by someone different from the subject 506 and/or different from the user. In some cases, the wearable device 532, may even comprise a vest or collar worn by a companion or service animal that is in proximity to the subject 506. The external control device 510 is configured for two-way communication (signals 538) with the wearable device 532, and the wearable device 532 is configured for two-way communication (signals 540) with the adjustable implants 502, 504. A transceiver 542 carried by the external control device 510 and a transceiver 544 carried by the wearable device 532 enable this communication. Thus, the wearable device 532 may serve as an intermediary device between the external control device 510 and the adjustable implants 502, 504.

In some embodiments, the user interface 536 if the wearable device 532 is configured to perform some or all of the operations of the GUI 512 of the external control device 510. In some embodiments, the wearable device 532 comprises memory 546 that is configured to store data from the adjustable implants 502, 504, form the subject 506, and/or from the external control device 510. Though the wearable device 532 is shown in FIG. 5 as a wrist-worn device, in other embodiments, the wearable device 532 may be configured to be worn on any part of the subject 506, and may comprise a hat, collar, article of clothing, or even a blanket. The wearable device 532 and the external control device 510 may be powered with rechargeable batteries, replaceable batteries, and may be chargeable by traditional contact methods or be non-contact (e.g., inductive) methods. The wearable device 532 and the external control device 510 may also be plugged into a power outlet.

The external control device 510, by means of the transceiver 542, or another transceiver, is configured for two-way communication (signals 548) with a cloud computing system 550. Data from the adjustable implants 502, 504 may be transmitted to the cloud computing system 550, and may be stored in the cloud computing system 550 for later use. In addition, information that is useful to the operation of the adjustable implants 502, 504 may be stored in the cloud computing system 550, and may be acquired by the external control device 510 from the cloud computing system 550. The information may include new adjustment settings, or operational codes or constants that affect the operation of the adjustable implants 502, 504. A computer 552 having a display 554 and a user interface 556 is located remotely, for example, in a physician's office, and is also configured for two-way communication (signals 558) with the cloud computing system 550. Thus, a physician controlling the treatment of the subject 506 may at any time change the prescription to the subject 506 by creating one or more new adjustment settings and transmitting, via transmitter 560 on the computer 552, the new adjustment settings to the cloud computing system 550. The prescription can at any time be altered by the physician remotely. The physician and the subject 506 (or the person responsible for the subject 506, such as a parent, friend, or family member) may each have a particular password or other entry code or process that allows them to access the data carried on the cloud computing system 550, related to the treatment of the subject 506. Via this security feature, the physician or other medical staff is thus capable of assessing from their office or other remote location, the latest status of the adjustable implants 502, 504 of the subject 506, and capable of making any changes to the treatment of the subject 506 that will be receivable by the external control device 510. Security codes may be entered by the user tactilely, or by voice. Other secure entry features may be employed that do not require entry of codes, such as fingerprint ID (tactile, optical, other), eye ID (iris identification, retinal scanning), other biological or physiological identifiers, or implanted chips (in the subject and/or in the user). Additionally, the physician or other medical staff may be able to receive reports that the patient is or is not being compliant with the planned treatment. The data may be voluntarily or automatically sent to additional locations, such as a provider main office or location, or a payer, such as a medical insurance company.

The data may be encrypted prior to moving to the cloud computing system 550 from any location. In some embodiments, direct two-way communication may exist between the cloud computing system 550 and the wearable device 532, via the transceiver 544. In some embodiments, direct two-way communication may exist between the cloud computing system 550 and the adjustable implants 502, 504, via the transceiver 21. Because of the existence of memory in all the mentioned locations, data can be stored in any of the adjustable implants 502, 504, the wearable device 532, the external control device 510, the cloud computing system 550, or the computer 552, or other computers in the physician's office or practice. In some cases, the active, real-time adjustment of the adjustable implants 502, 504 may be controlled remotely on the computer 552 from the physician's office. The physician or medical personnel may choose to communicate by telephone with the subject 506 or someone responsible for the subject 506 during the remote adjustment, but in other embodiments, a speaker/microphone system on the computer 552 or in the physician's office, and on the external control device 510 or wearable device 532 may preclude the use of the telephone. The external control device 510 or wearable device 532, thus, may themselves be smartphones having their own telephone numbers. The standard adjustment settings may include the number of adjustments per day, per week, per month, or per year, and the adjustment length per adjustment. The length per adjustment or the number of adjustments per time may be automatically scaled to increase or decrease over time.

In some embodiments, the systems for adjusting tissue in a patient 1, 500 may incorporate artificial intelligence (AI). For example, in step 210 of the method 200 of FIG. 2B, the growing rod 10 sends a progress of the adjustment to the external control device 5, and in step 114 of the method 100 of FIG. 2A, the external control device 5 receives a progress of the adjustment from the selected growing rod 10. Following either of these steps, the data may be manipulated with artificial intelligence (AI) to modify, change, tailor, or restructure the operation of the systems for adjusting tissue in a patient 1, 500. For example, the data may be manipulated with artificial intelligence (AI) to at least partially include the creation of a modified adjustment setting. The modified adjustment setting may have an increased or decreased distraction or compression length, or an increased or decreased distraction or compression rate (longitudinal velocity), or an increased or decreased target force measurement. The modified value may be determined automatically with AI via software algorithms that factor in response factors from the prior completed adjustments using prior adjustment settings. For example, a ratio of distraction length change to force measurement by the sensor 17 may be used by the AI to determine whether to scale subsequent distraction lengths up or down. For example, all subsequent calculated target distraction lengths may be multiplied by an additional factor of 1.1, to scale up, or by 0.9 to scale down, or multiplied by 1.2 to scale up, or by 0.8 to scale down. Alternatively, or in additional, the number of adjustments per time may be increased or decreased. For example, based on the ratio of distraction length change to force measurement by the sensor 17 in the prior adjustments, the AI may divide what would previously have been five lengthenings at two millimeters each over eight months, to ten lengthenings at one millimeter each over the same eight months. The AI may also incorporate other data, such as the Cobb angle in the scoliotic curve measured after the adjustment, or the change in Cobb angle from before the adjustment to after the adjustment. The AI may also incorporate the age of the subject, the height or sitting height, or other maturity factors, such as Risser sign to automatically calculate how to scale the modified adjustment setting. Statistics may be inserted into algorithms to provide machine learning that allows the adjustment settings to modified throughout the life of the implantation of and treatment by the growing rod/adjustable implant 10, 502, 504. The physician may adjust a level of control so that approval of the physician is required for each AI or machine learning-derived change in the adjustment setting, or so that approval is not required. The physician will be able, if desired, to begin treatment requiring physician approval for each modification in adjustment setting, and then after a sufficient time of acceptable treatment, removing the approval requirement to allow the software to make the changes automatically and autonomously. The growing rod/adjustable implant 10, 502, 504 may thus be configurable to be automatically adjusted via control from the cloud computing system 550.

Returning to FIG. 5, a feedback loop may be created between the adjustable implants 502, 504 and any one or more of the controlling devices 510, 532, 552 such that new adjustment settings are automatically generated based on changes in measured force, or changes in measured force with respect to time. The feedback loop may even be triggered by an algorithm that is stored on the cloud computing system 550, as data travels from one or more of the adjustable implants 502, 504 to the cloud computing system 550. Data moving in either direction through the cloud computing system 550 may utilize an application programming interface (API) to and from hospital data platforms or electronic health records.

As will be appreciated by one skilled in the art, aspects of the present disclosure may be embodied as a system, method or computer program product. Accordingly, aspects of the present disclosure may take the form of an entirely hardware-based embodiment, an entirely software-based embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable storage medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM), Flash memory, an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer storage medium does not include propagating signals.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object-oriented programming (OOP) language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Network using a Network Service Provider). Any of the components of the system in the embodiments of the present disclosure described herein may be configured to communicate with a cloud computing system.

Aspects of the present disclosure are described herein with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

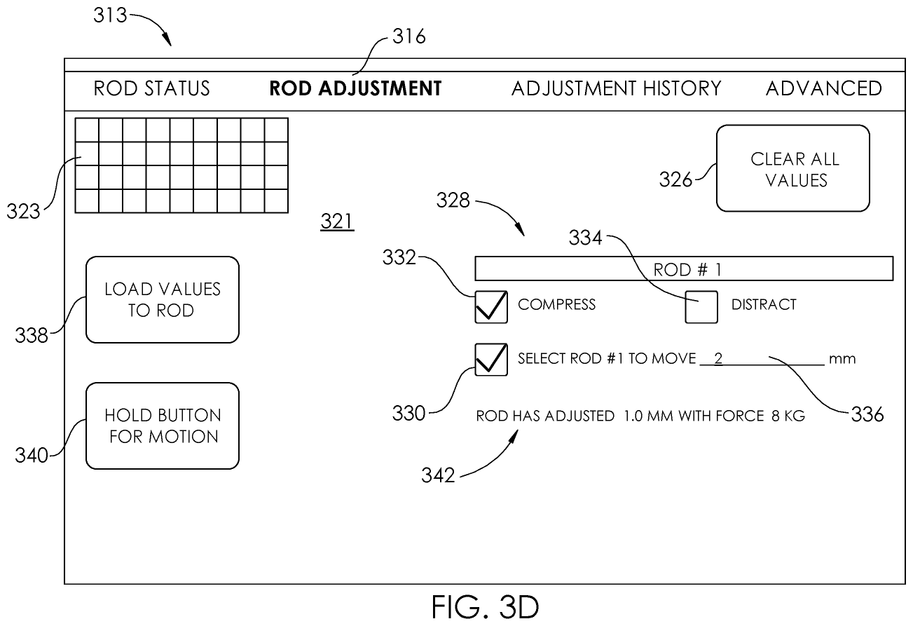

Adjustable implants comprising motors have generally been presented in the previous embodiments. The motors 8, 526, 528 may comprise an electric motor, such as a brushed or brushless motor, a DC motor, a stepper motor, a servo motor, a linear motor, or an ultrasonic or piezo motor, including an inertia motor, a resonance motor, or a piezo-walk drive. FIGS. 6-9 schematically illustrate four alternate embodiments. FIG. 6 illustrates a system for adjusting tissue in a patient 1300 comprising an adjustable implant 1306 having a first implant portion 1302 and a second implant portion 1304, the second implant portion 1304 is non-invasively displaceable in relation to the first implant portion 1302. The first implant portion 1302 is secured to a first bone portion 197 and the second implant portion 1304 is secured to a second bone portion 199 within a patient 191. A rotatable magnet 1308 is operable to cause the first implant portion 1302 and the second implant portion 1304 to displace relative to one another. An external control device 1310 has a control panel 1312 for input by an operator, a display 1314 and a transmitter 1316. The transmitter 1316 produces a changing magnetic field 1318 through the skin 195 of the patient 191 to cause the magnet 1308 to rotate, driving an internal drive train (e.g., lead screw) that causes the first implant portion 1302 and the second implant portion 1304 to displace relative to each other. The transmitter 1316 may comprise one or more electromagnetic coils, but in alternative embodiments, may comprise one or more rotating permanent magnets. A transceiver 1320 may be coupled to the adjustable implant 1306 by a wire 1322 and may be configured to communicate with the external control device 1310, to transfer information bidirectionally between the external control device 1310 and the adjustable implant 1306. Either the rotatable magnet 1308 or the alternative rotating permanent magnet(s) of the external control device 1310 may comprise cylindrical magnets that are radially poled.

FIG. 7 illustrates a system for adjusting tissue in a patient 1400 comprising an adjustable implant 1406 having a first implant portion 1402 and a second implant portion 1404, the second implant portion 1404 non-invasively displaceable in relation to the first implant portion 1402. The first implant portion 1402 is secured to a first bone portion 197 and the second implant portion 1404 is secured to a second bone portion 199 within a patient 191. An ultrasonic motor 1408 is operable to cause the first implant portion 1402 and the second implant portion 1404 to displace relative to one another. An external control device 1410 has a control panel 1412 for input by an operator, a display 1414 and an ultrasonic transducer 1416, which is coupled to the skin 195 of the patient 191. The ultrasonic transducer 1416 produces ultrasonic waves 1418 which pass through the skin 195 of the patient 191 and operate the ultrasonic motor 1408.

FIG. 8 illustrates a system for adjusting tissue in a patient 1700 comprising an adjustable implant 1706 having a first implant portion 1702 and a second implant portion 1704, the second implant portion 1704 non-invasively displaceable in relation to the first implant portion 1702. The first implant portion 1702 is secured to a first bone portion 197 and the second implant portion 1704 is secured to a second bone portion 199 within a patient 191. A shape memory actuator 1708 is operable to cause the first implant portion 1702 and the second implant portion 1704 to displace relative to one another. An external control device 1710 has a control panel 1712 for input by an operator, a display 1714 and a transmitter 1716. The transmitter 1716 sends a control signal 1718 through the skin 195 of the patient 191 to an implanted receiver 1720. Implanted receiver 1720 communicates with the shape memory actuator 1708 via a conductor 1722. The shape memory actuator 1708 may be powered by an implantable battery, or may be powered or charged by inductive coupling.

FIG. 9 illustrates a system for adjusting tissue in a patient 1800 comprising an adjustable implant 1806 having a first implant portion 1802 and a second implant portion 1804, the second implant portion 1804 non-invasively displaceable with relation to the first implant portion 1802. The first implant portion 1802 is secured to a first bone portion 197 and the second implant portion 1804 is secured to a second bone portion 199 within a patient 191. A hydraulic pump 1808 is operable to cause the first implant portion 1802 and the second implant portion 1804 to displace relative to one another. An external control device 1810 has a control panel 1812 for input by an operator, a display 1814 and a transmitter 1816. The transmitter 1816 sends a control signal 1818 through the skin 195 of the patient 191 to an implanted receiver 1820. The implanted receiver 1820 communicates with the hydraulic pump 1808 via a conductor 1822. The hydraulic pump 1808 may be powered by an implantable battery, or may be powered or charged by inductive coupling. The hydraulic pump 1808 may alternatively be replaced by a pneumatic pump.

In any of the systems for adjusting tissue in a patient 1300, 1400, 1700, 1800 the receivers 1720, 1820 may comprise transceivers. Furthermore, the transceivers 1320 or receivers 1720, 1820 may be external to the adjustable implant 1306, 1406, 1706, 1806, or may be located on or in the adjustable implant 1306, 1406, 1706, 1806.

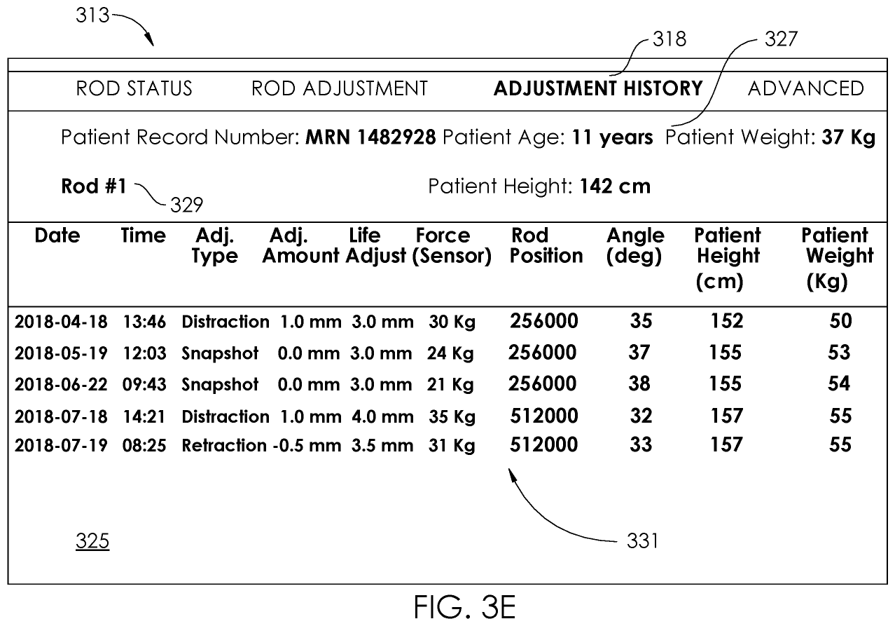

Systems comprising adjustable implants, such as the embodiments disclosure herein, may be utilized to treat a number of different maladies, including, but not limited to early onset scoliosis, adolescent idiopathic scoliosis, limb-length discrepancy, cranio-maxillofacial deformity, soft tissue tightness, or soft tissue laxity, including the adjustment of ligaments, such as the ACL, or muscles, such as eye muscles in a strabismus patient, or tendons and muscles, such as the rotator cuff. Different bones or portions of bones may be lengthened, shortened, distracted, compressed, curved, uncurved, rotated, derotated, and otherwise reshaped, reoriented, or reformed, using the systems described herein. An adjustable implant 700, as shown in FIG. 10, is fully implantable in the medullary canal 714 of a patient's bone 704. The adjustable implant 700 is shown implanted in a femur having a first portion 705 and a second portion 707, separated by a natural fracture or purposeful osteotomy 709. The adjustable implant 700 includes an internally mounted power supply 730, an internally mounted electronic control/communication assembly 732 (e,g, controller and transceiver) a motor 734, a gear train 735, which drive a drive screw 724, to longitudinally translate a first end 711 in relation to a second end 713 of the adjustable implant 700. The two ends 711, 713 include transverse holes 737, 739 through which pins or screws 718, 720 may be placed to secured the adjustable implant 700 to the second portion 707 and first portion 705 of the bone 704. By distracting at a controlled rate (e.g., one millimeter per day, or ⅓ millimeter each eight ours), bone can be grown at the osteotomy 709 while the distraction process occurs, and after the distraction process is completed. After the completion of the distraction process, the adjustable implant 700 may be used to compress the first portion 705 and the second portion 707 of the bone 704 together, if desired, to potentially accelerate the healing and solidification process of the bone 704 (femur).

An adjustable implant 750 having a first portion 752 and a second portion 724 is configured to adjust the length of a tibia 756 having a first portion 758 and a second portion 760, separated by a fracture or purposeful osteotomy 762. The fibula 764 may also be divided into a first portion 766 and second portion 768 with a fracture or purposeful osteotomy 770. The adjustable implant 750 may be attached to one or more plate 772 configured to attach to an outer portion of the tibia 756, and may be secured to the tibia 756 with pins or screws 774. The adjustable implant 750 may include any of the internal components and operation of the other embodiments of adjustable implant described herein. By distracting at a controlled rate (e.g., one millimeter per day, or ⅓ millimeter each eight ours), bone can be grown at the osteotomy(ies) 765, 770 while the distraction process occurs, and after the distraction process is completed. After the completion of the distraction process, the adjustable implant 750 may be used to compress the first portion 758 and the second portion 760 of the tibia 756 together, if desired, to potentially accelerate the healing and solidification process of the tibia 756.

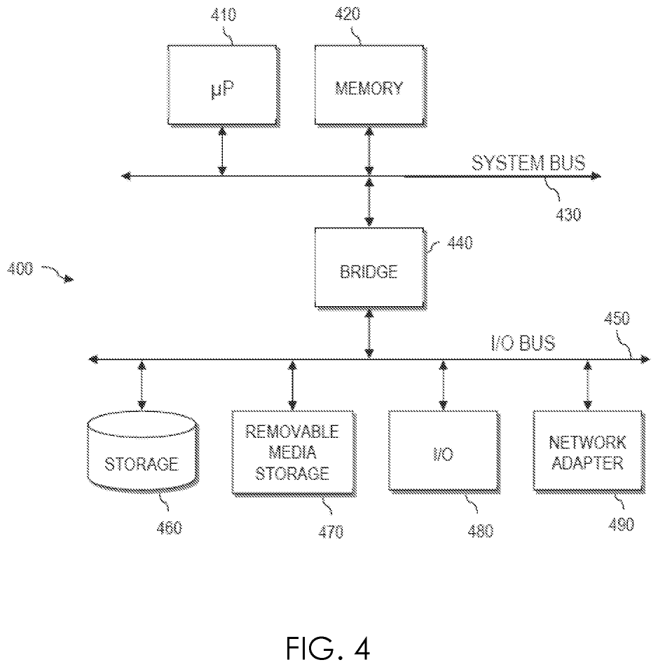

FIGS. 12-13 illustrates using adjustable implants with wedge osteotomies to adjust the angle or curvature of a bone. FIG. 12 schematically shows a frontal view of a right femur 600 exhibiting a curvature deviating from the natural form of this bone. The curvature may be due to a congenital disease or other condition. The dashed lines 601, 602 indicate how the bone can be fractured, by sawing or other methods, to form wedge osteotomies. In one example, wedge shaped portions of bone are removed and the bone divided into sections, here illustrated as three sections. FIG. 13 shows how these three sections of the femur 603 are repositioned to a desired orientation, i.e. a straighter bone. The fracture zones 604, 605 are then used as growth zones in order to compensate for the loss of length due to the removal of bone. Adjustable implants 606, 607 are then attached via anchors (pins, screws, etc.) to said sections, ensuring their position. By lengthening at a different rate on one side of the femur 603 from the other side of the femur 603, the adjustable implants 606, 607 are able to apply a moment or rotate the bone along a two different transverse axes, thus straightening the femur 603 by distractive osteogenesis. In other cases, an undesirably straight bone can be caused to increase its curvature, by a similar, but opposite method. The arrows illustrate schematically that the parts of the bone can be adjusted in relation to each other, for example by adjusting the angle or orientation of said parts. According to another embodiment, two or more anchoring devices are adapted to engage the cortical part of the bone. According to another embodiment, said two or more anchoring devices are adapted to engage the bone from the inside of the medullary cavity. According to another embodiment, said at least two anchoring devices are chosen from a pin, a screw, an adhesive, a barb construction, a saw-tooth construction, an expandable element, combinations thereof or other mechanical connecting members. According to a further embodiment, the force exerted by the adjustment device is a longitudinal force, extending the length of the bone. According to an embodiment, said force exerted by the adjustment device is directed to the end portions of the medullar cavity. According to an embodiment, said the force exerted by the adjustment device is a longitudinal force, adjusting the angle or curvature of the bone. According to an embodiment, said the force exerted by the device also applies at least some torque to the bone, adjusting the torsion of the bone along its longitudinal axis.

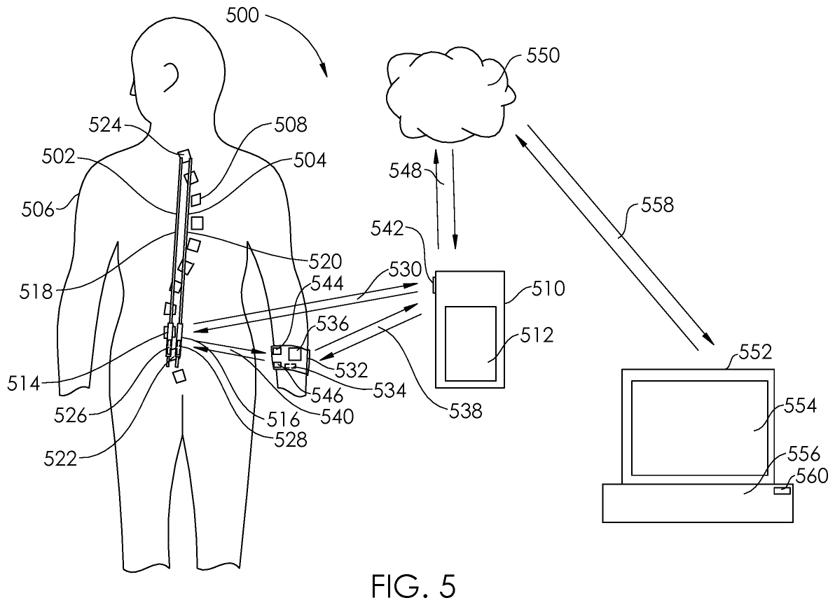

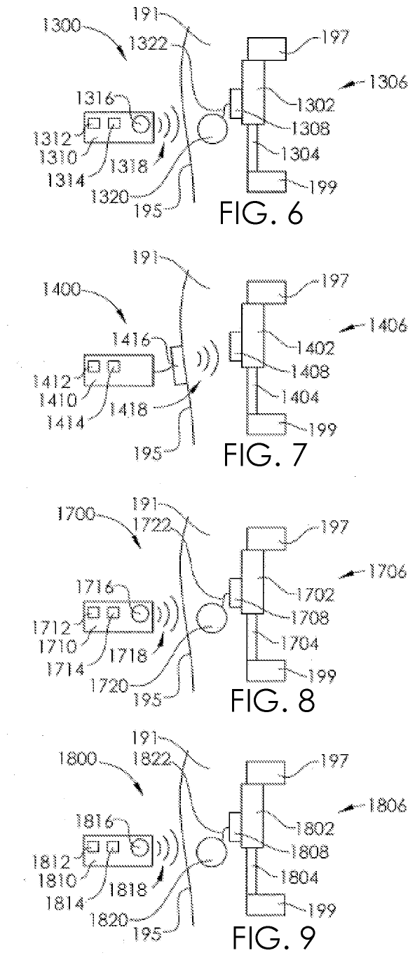

A related embodiment is illustrated in FIGS. 14 and 15, where a deformed bone 600 is cut at two locations, 601 and 602, each cut preferably being wedge shaped in order to allow for the straightening of the bone, and adjustable implants 610 and 620 are inserted into the medullar cavity. Similarly, as in FIG. 13, the arrows illustrate schematically that the parts of the bone can be adjusted in relation to each other, for example by adjusting the angle or orientation of said parts. According to yet another embodiment, the force exerted by the device applies torque to the bone, adjusting the torsion of the bone along its longitudinal axis. This embodiment is illustrated in FIGS. 16 and 17, where a bone 600 is cut along the dashed line 630 and optionally along one or more lines, exemplified as 631. One or more adjustable implants 640 and 650 are inserted into the medullar cavity. The arrows indicate that one or several parts of the bone can be adjusted, for example rotated in relation to a joint, or to a section of the bone. According to yet another embodiment, freely combinable with any of the embodiments presented herein, said device is flexible to allow introduction into the medullar cavity. Any of the adjustable implants 700, 750, 606, 607, 610, 620 may comprise some of all of the elements of the adjustable implants 10, 502, 504 described in more detail herein, and may be used and controlled in conjunction with the systems 1, 500 described herein. The adjustable implants 640, 650 are contemplated to be motorized versions (having internal motors instead of internal magnets) of the intramedullary rotational correction devices described in in U.S. Pat. No. 8,715,282, entitled “System and Method for Altering Rotational Alignment of Bone Sections,” issued May 6, 2014, which is hereby incorporated by reference in its entirety for all purposes.

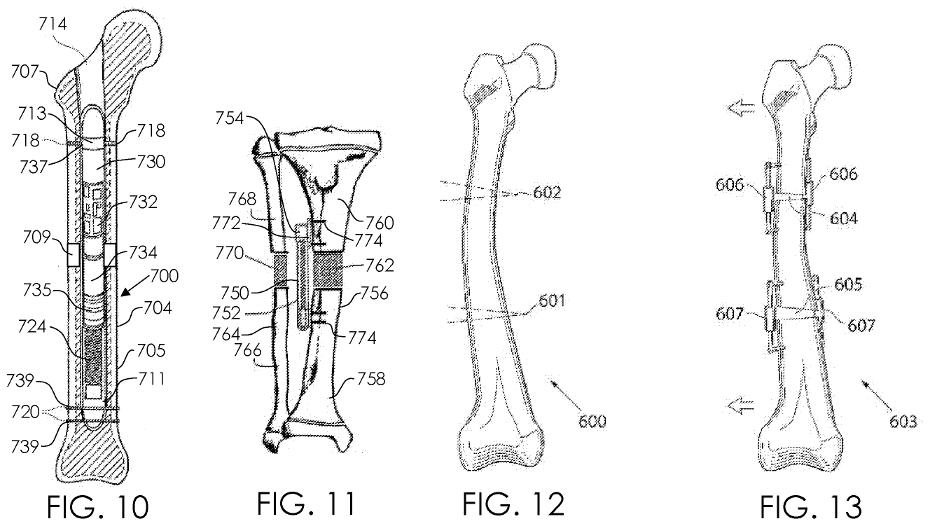

FIG. 18 illustrates a skull 780 having several osteotomies 782 which were formed in order to adjust the cranio-facial anatomy. Several adjustable implants 784 have been secured to two different adjacent portions of bone on both sides of the osteotomies 752. The adjustable implants 784 may comprise some of all of the elements of the adjustable implants 10, 502, 504 described in more detail herein, and may be used and controlled in conjunction with the systems 1, 500 described herein. The adjustable implants 784 are configured to distract or contract different portions of the cranio-facial bone to reform the shapes and sizes of areas of the face using distraction osteogenesis. This reshaping may include jaw (mandibular) lengthening in patient having micrognathia.

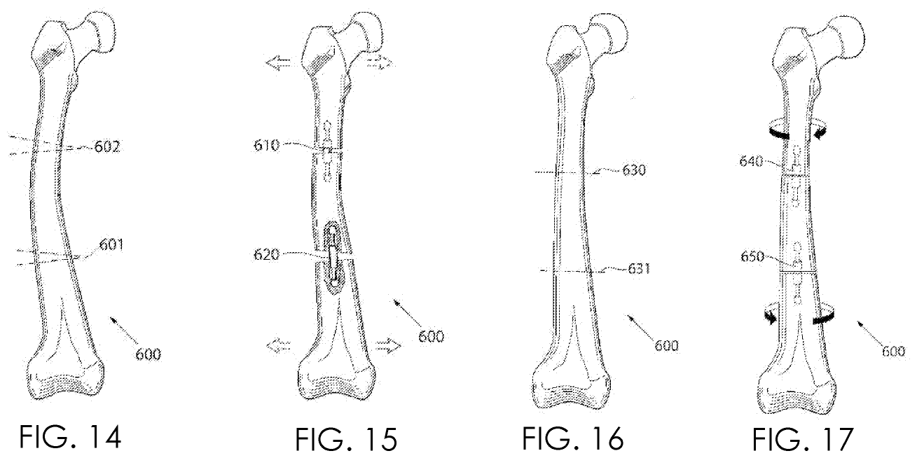

The adjustable implants 10, 502, 504 and the systems for adjusting them 1, 500 described herein may also be configured to adjust soft tissue, as previously described. In FIG. 19, a glenohumeral (shoulder) joint 852 is illustrated, and includes a rotator cuff 854 and a humerus 860 having a greater tubercle 862 and a humeral head 864. An adjustable suture anchor 800 has a first end 802 and a second end 804. A loop of suture 816 extends from a tendon 850 (connecting a muscle 851) in an external portion 871 and an internal portion 872. A tunnel 874 through which the suture 816 can slide is made in the tendon 850, so that the length of the loop of suture 816 which extends from point A to point B to point C, can be adjusted, thus adjusting the tension with which the suture 816 holds the tendon 850. A pad 876 of biocompatible material is placed underneath the suture 816 to minimize damage to the tendon as the suture 816 slides over it. A first end 802 of the adjustable suture anchor 800 includes a threaded portion 812 and an external circumferential groove 878, around which external portion 871 of suture 816 can be wrapped and/or tied. A second end 804 of the adjustable suture anchor 800 has a tapered tip 808, which aids its insertion. Within a longitudinal cavity 836 of the housing 810 of the adjustable suture anchor 800, a motor 841 is held, the motor 841 configured to rotate a rotating shaft 880. A spool 822 is secured to the shaft 880 so that rotation of the shaft 880 causes rotation of the spool 822. A seal or diaphragm 852 is carried within an aperture 882 in the lateral wall of the housing 810, allowing the internal portion 872 of the loop of suture 816 to move in and out of the housing 810 of the adjustable suture anchor 800, with the contents of the longitudinal cavity 836 remaining protected from body fluids.

During implantation, two pilot holes are drilled through which through the cortical bone 856 and cancellous bone 858, including a first hole 866 extending from point C toward point A. The first hole 866 may even be extended to create an additional pocket 868. A second hole 870 extends from point B towards (and just past) point A. A grasper tool is placed through hole 870, and a suture insertion tool inserts the end of the external portion 871 of the suture 816 through first hole 866. The grasper tool grasps the suture 816 and pulls it out through second hole 870. The adjustable suture anchor is then inserted and secured inside first hole 866, tightening it with a driving tool inserted into a keyed cavity 814. The housing may be oriented so that the aperture 882 extends in a direction towards second hole 870. The external portion 871 of the suture 816 is now placed through the tunnel 874 in the tendon 850, and then wrapped and/or tied around the external circumferential groove 878, thus closing the loop in the suture 816. To adjust the tension of the suture 816, the system 1, 500 is operated to cause the motor 841 to turn and the shaft 880 and spool 822, to tighten the tension in the suture 816, and thus in the tendon 850. The motor 841 may be operated in an opposite rotational direction in order to loosen the tension in the suture 816 and tendon 850.

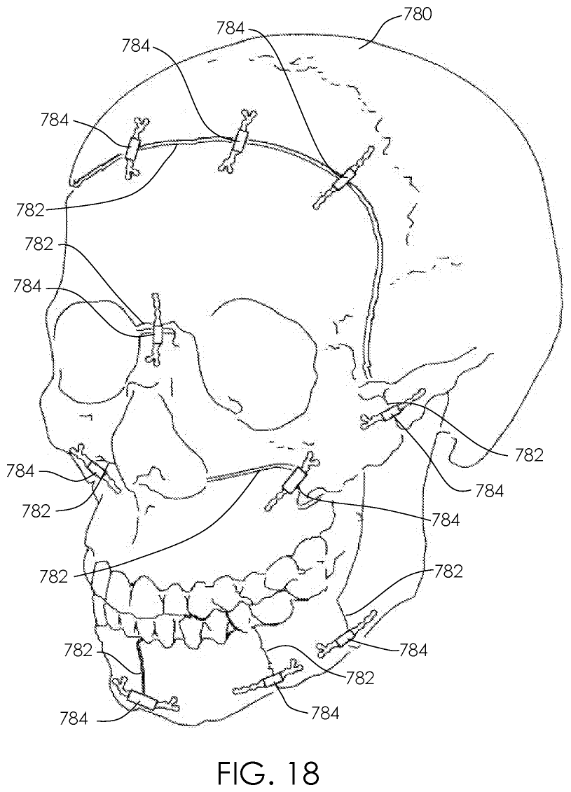

FIG. 20 illustrates an alternative geometry for creating a hole 884 at the greater tubercle 862 of the humerus 860. An adjustable suture anchor 900 having an adjustable component 922 is implanted in the hole 884 and is capable of adjusting the tension in a suture 916, which is attached to a tendon 850 of a rotator cuff 854. The hole 884 is parallel the axis of the humerus 860, thus allowing for a longer length adjustable suture anchor 900 to be utilized. This makes possible an adjustable suture anchor 900 containing more planetary gear sets and allow allows for a greater range of adjustability (length, tension).

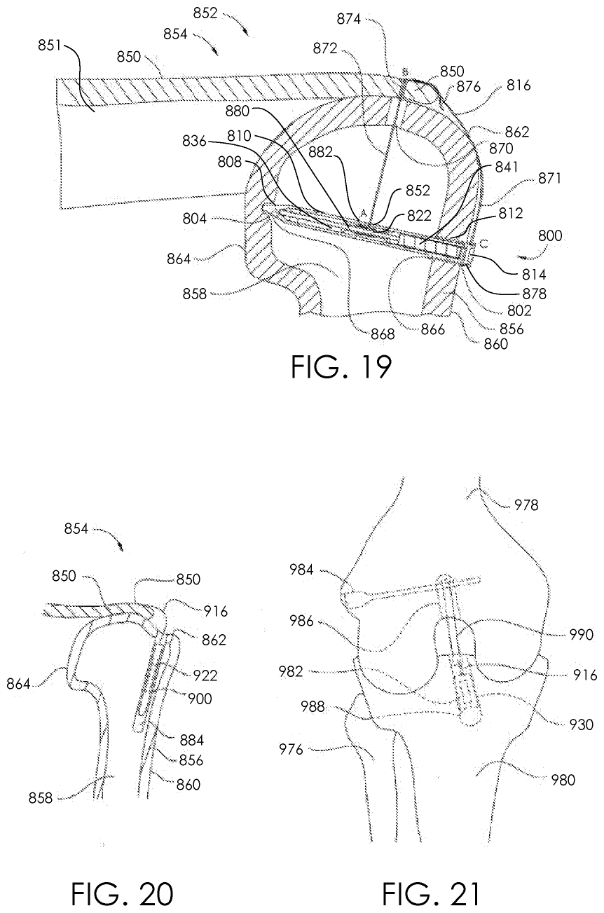

Though the adjustable suture anchors 800,900 as described are adapted for attaching the tendon of the rotator cuff to the humerus, it is conceived that similar suture anchors would be useful for adjusting other soft tissue attachments to bone. Some examples include the anterior cruciate ligament (ACL) in one or both of its attachment point to the bone (femur and/or tibia). FIG. 21 shows a configuration for an adjustable suture anchor 930 for adjusting the tension in a graft 990 for replacing the ACL (for example, the graft may be a portion of the patellar tendon of the patient that is cut or dissected out for use). The graft 990 is secured in a femoral tunnel 986 in a femur 978 with a traditional tissue anchor 984. The fibula 976 is also shown, as a reference. The tissue anchor 984 may be metallic, or may be of a resorbable material. The adjustable suture anchor 930 is anchored to bone inside a tibial tunnel 988 created in a tibia 980. A motor 982 within the adjustable suture anchor 930 adjusts the tension in a suture 916 which is attached to the graft 990. The diameter of the tissue anchor 984 may be less than about 14 mm, or less than about 12 mm. The length of the femoral tunnel 986 may be on the order of about 25 mm to about 35 mm.

To adjust the tension of the suture 916, the system 1, 500 is operated to cause the motor 982 to turn, for example, a shaft and spool (not shown) to tighten the tension in the suture 916, and thus in the graft 990. The motor 982 may be operated in an opposite rotational direction in order to loosen the tension in the suture 916 and graft 990.

Other types of soft tissue adjustment may utilize similar adjustable implants as those described in the previous embodiments. In some embodiments, the adjustable implants may be configured to secure to at least one bony structure and at least one non-bony structure. In some embodiments, the adjustable implants may be configured to secure to at least two non-bony structures. In some embodiments, the adjustable implants may be configured for adjusting eye muscles in a strabismus patient. In some embodiments configured for treatment of strabismus, a first end of the adjustable implant may be configured to secure to a first portion of an eye muscle and a second end of the adjustable implant may be configured to secure to a second portion of the eye muscle (e.g., extraocular or rectus muscles). The adjustable implant may be partially or completely imbedded in the sys muscle, or may be external or mostly external to the eye muscle, but because it is at least partially within the eye socket, it can still be considered an “implant.”

In one embodiment, a method for adjusting an adjustable implant in a subject includes providing a device having a graphical user interface, wirelessly detecting one or more adjustable implants, selecting at least one of the one or more adjustable implants with the graphical user interface, sending an adjustment setting to the at least one of the one or more adjustable implants, and sending an instruction to the at least one of the one or more adjustable implants to perform the adjustment setting. In some embodiments, the method comprises instructions stored on a non-transitory computer-readable medium. In some embodiments, the instructions, when executed on a processor, perform the steps of the method. In some embodiments, the instructions, when executed on a processor, activate a motor in the adjustable implant. In some embodiments, the non-transitory computer-readable medium is carried by the device. In some embodiments, the processor is carried on the device, and the non-transitory computer-readable medium is executable on the processor via input by a user to the graphical user interface. In some embodiments, the device is configured to communicate information to a user visually. In some embodiments, the device is configured to communicate information to a user audibly. In some embodiments, the device is configured to communicate information to the user via a computer-controlled or computer-generated voice. In some embodiments, the device is configured to communicate information to the user via a recorded voice. In some embodiments, the graphical user interface is configured to receive input from a user tactilely. In some embodiments, the device is configured to receive input from a user via the user's voice. In some embodiments, sending an instruction to the at least one of the one or more adjustable implants to perform the adjustment setting comprises sending the adjustment setting with the graphical user interface. In some embodiments, the method further comprises receiving a progress of the adjustment setting from the at least one of the one or more adjustable implants. In some embodiments, the adjustable implant comprises a first portion configured to couple to a first location in the subject, a second portion moveably coupled to the first portion and configured to couple to a second location in the subject, and a motor configured to move the first portion and the second portion relative to each other. In some embodiments, the adjustable implant is configured to move at least a first vertebra of the subject in relation to a second vertebra of the subject. In some embodiments, the adjustable implant is configured to move at least a first bone of the subject in relation to a second bone of the subject. In some embodiments, the adjustable implant is configured to move at least a first portion of a separated bone of the subject in relation to a second portion of the separated bone of the subject. In some embodiments, the adjustable implant is configured to apply tension or compression on soft tissue of the subject.

In some embodiments, the adjustment setting comprises a distraction direction (e.g., an increase in length, or +Z or a decrease in length, or −Z). In some embodiments, the adjustment setting comprises a length (e.g., 1 mm of length added during a distraction, or 15 mm total distracted length to attain as a result of the distraction). In some embodiments, the adjustment setting comprises a target distraction force or target compression force. In some embodiments, sending an instruction to the at least one of the one or more adjustable implants to perform the adjustment setting comprises receiving, via the graphical user interface, an instruction to start the adjustment setting. In some embodiments, sending an instruction to the at least one of the one or more adjustable implants to perform the adjustment setting comprises receiving, via the graphical user interface, an instruction to pause the adjustment setting. In some embodiments, sending an instruction to the at least one of the one or more adjustable implants to perform the adjustment setting comprises receiving, via the graphical user interface, an instruction to restart the adjustment setting. In some embodiments, sending an instruction to the at least one of the one or more adjustable implants to perform the adjustment setting comprises receiving, via the graphical user interface, an instruction to terminate the adjustment setting. In some embodiments, receiving a progress of the adjustment setting from the at least one of the one or more adjustable implants comprises receiving an indication from the adjustable implant that the adjustment is complete. In some embodiments, the method comprises receiving from the adjustable implant information about the adjustable implant and displaying at least some of the information on the graphical user interface. In some embodiments, the method comprises receiving from the adjustable implant information associated with the subject and displaying at least some of the information on the graphical user interface. In some embodiments, the information about the adjustable implant comprises at least one of: a history of adjustments previously made on the adjustable implant, a previous length of the adjustable implant, a current length of the adjustable implant, a previous distraction force or compression force of the adjustable implant, a current distraction force or compression force of the adjustable implant, a motor temperature, a motor duty cycle time, a battery charge level, or a clock time. In some embodiments, the information associated with the subject comprises at least one of: a height of the subject, a sitting height of the subject, a weight of the subject, a birth date of the subject, the age of menarche of the subject, a Cobb angle of the subject, or other demographic information about the subject.

In some embodiments, wirelessly detecting one or more adjustable implants comprises receiving an identity of the one or more adjustable implants via a network, wherein the one or more adjustable implants are remotely located. In some embodiments, wirelessly detecting one or more adjustable implants comprises receiving an identity of the one or more adjustable implants via an intermediary device, wherein the one or more adjustable implants are remotely located. In some embodiments, wirelessly detecting one or more adjustable implants comprises receiving a list of adjustable implants. In some embodiments, selecting at least one of the one or more adjustable implants with the graphical user interface comprises receiving a selection of the adjustable implants on the graphical user interface, the selection resulting from the list of adjustable implants.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the invention in the form disclosed. Many modifications and variations will be apparent to those of ordinary skill in the art without departing from the scope and spirit of the invention. Aspects of the disclosure were chosen and described in order to best explain the principles of the invention and the practical application, and to enable others of ordinary skill in the art to understand the invention for various embodiments with various modifications as are suited to the particular use contemplated.

1. A method of adjusting an adjustable implant, the method comprising:
broadcasting identification information from an adjustable implant implanted within a subject;
receiving wirelessly with the adjustable implant, from a device external to the subject, an adjustment setting configured to be executed by the adjustable implant;
receiving wirelessly with the adjustable implant, from a device external to the subject, an instruction to perform the adjustment setting;
activating a motor on the adjustable implant to perform the adjustment setting; and
sending, to a device external to the subject, a progress of the adjustment setting.

2. The method of claim 1, wherein the adjustment setting is received from a first device and the instruction to perform the adjustment setting is received from a second device.

3. The method of claim 2, wherein the progress of the of the adjustment is sent to the second device.

4. The method of claim 2, wherein the progress of the adjustment is sent to a third device.

5. The method of claim 1, wherein adjustment setting and the instruction to perform the adjustment setting are each received from a first device.

6. The method of claim 5, wherein the progress of the adjustment is sent to the first device.

7. The method of claim 1, further comprising:
storing the adjustment setting in a database on the adjustable implant; and
storing the progress of the adjustment setting in the database on the adjustable implant.

8. The method of claim 1, wherein receiving wirelessly with the adjustable implant, from a device external to the subject, an instruction to perform the adjustment setting comprises receiving an instruction to start the adjustment setting, and wherein activating a motor on the adjustable implant to perform the adjustment setting comprises initiating the motor on the adjustable implant.

9. The method of claim 8, wherein receiving wirelessly with the adjustable implant, from a device external to the subject, an instruction to perform the adjustment setting comprises receiving an instruction to pause the adjustment setting, and wherein activating a motor on the adjustable implant to perform the adjustment setting comprises stopping the motor on the adjustable implant.

10. The method of claim 9, wherein receiving wirelessly with the adjustable implant, from a device external to the subject, an instruction to perform the adjustment setting comprises receiving an instruction to resume the adjustment setting, and wherein activating a motor on the adjustable implant to perform the adjustment setting comprises restarting the motor on the adjustable implant.

11. The method of claim 1, wherein sending, to a device external to the subject, a progress of the adjustment setting comprises sending an indication that the adjustment setting has completed.

12. The method of claim 1, wherein the adjustable implant comprises:
a first portion configured to couple to a first location in the subject;
a second portion moveably coupled to the first portion and configured to couple to a second location in the subject; and
wherein the motor is configured to move the first portion and the second portion relative to each other.

13. The method of claim 12, wherein the adjustable implant is configured to move at least a first vertebra of the subject in relation to a second vertebra of the subject.

14. The method of claim 12, wherein the adjustable implant is configured to move at least a first bone of the subject in relation to a second bone of the subject.

15. The method of claim 12, wherein the adjustable implant is configured to move at least a first portion of a separated bone of the subject in relation to a second portion of the separated bone of the subject.

16. The method of claim 12, wherein the adjustable implant is configured to apply at least one of tension or compression on soft tissue of the subject.

17. The method of claim 1, wherein the adjustment setting comprises at least one of a distraction direction or a distraction length.

18. The method of claim 1, wherein the progress of the adjustment setting sent to the device external to the subject comprises at least one of a length or a force.

19. The method of claim 1, further comprising:
after sending, to the device external to the subject, the progress of the adjustment setting, using artificial intelligence (AI)-learning to at least partially influence the creation of a modified adjustment setting.

20. The method of claim 1, wherein the adjustable implant is configured to treat at least one ailment in the subject, chosen from the list consisting of: early onset scoliosis, adolescent idiopathic scoliosis, limb-length discrepancy, cranio-maxillofacial deformity, soft tissue tightness, or soft tissue laxity.

21-37. (canceled)
